(19) United States
(12) Patent Application Publication
(10) Pub. No.: US 2026/0306685 A1
(43) Pub. Date: Oct. 8, 2026

(54) POINT-OF-USE SYSTEM FOR MEDICAL INSTRUMENTS

(71) Applicant: BON SECOURS MERCY HEALTH, INC., Cincinnati, OH (US)

(72) Inventors: Namchi Phan Khac Le, Dayton, OH (US); Meghan Kieninger, Cumming, GA (US); Craig Ovans, Chandler, AZ (US); Matthew Ibarra, Cerritos, CA (US)

(21) Appl. No.: 19/483,038

(22) PCT Filed: May 8, 2024
(86) PCT No.: PCT/US2024/028234

§ 371 Date: Nov. 10, 2025

Related U.S. Application Data
(60) Provisional application No. 63/464,854, filed on May 8, 2023.

(51) Int. Cl. A61L 2/18; A61L 2/22; A61L 103/15

(52) U.S. Cl. CPC A61L 2/18; A61L 2/22; A61L 2103/15; A61L 2202/121; A61L 2202/122; A61L 2202/14; A61L 2202/17

(57) ABSTRACT

A system and method for cleaning a medical instrument are provided. The system can include a housing, a sanitizing drawer, a fluid reservoir, a spray nozzle, and a biohazard container. The sanitizing drawer can be disposed in the housing and can accept the medical instrument. The sanitizing drawer can open and close upon activation of a motion sensor disposed on the housing. The fluid reservoir can be disposed in the housing and can be configured to output a cleaning fluid to the sanitizing drawer. The spray nozzle can be disposed in the housing and can be configured to activate automatically after the sanitizing drawer is closed. The biohazard container can be removably disposed in the housing and can be positioned adjacent the sanitizing drawer. The biohazard container can be configured to accept the medical instrument from the sanitizing drawer automatically after the medical instrument is precleaned.

### CROSS-REFERENCE TO RELATED APPLICATIONS

This application claims the benefit of U.S. Provisional Application No. 63/464,854, filed on May 8, 2023. The entire disclosure of the above application is incorporated herein by reference.

### FIELD

The present technology relates to systems and methods for cleaning medical instruments and, more specifically, to automation of point-of-use processing or “precleaning” of reusable medical devices and instruments.

### INTRODUCTION

This section provides background information related to the present disclosure which is not necessarily prior art.

During medical procedures such as surgery, instruments may get contaminated with various bioburden such as blood, bone, tissue, inorganic soils such as dirt and dust, microorganisms, bacteria, viruses, and chemicals. Reusable instruments and other reusable devices must be effectively processed, including cleaning and sterilizing steps, in order to be safe and functional in preparation for the next patient use case. Also, if materials dry or bake onto the instruments, it becomes significantly more difficult to remove such bioburden from the instruments which may render subsequent cleaning and sterilization processes less effective or ineffective. Reusable medical instruments and devices require an additional step of precleaning before the primary sterilization process to mitigate the damage and risk that can be created by additional bioburden growth prior to the cleaning and sterilization process.

Point-of-use or pre-cleaning can occur by a few different industry accepted methods, including use of an enzymatic spray, pre-soaking, and maintaining a particular environment to prevent the development and hardening/attachment of bioburden to the instrument. Before an instrument can go through sterilization or high-level disinfection, it must be precleaned. The cleaning process requires consistency and standardization to ensure a quality outcome for the patient.

Currently, the pre-cleaning process is done manually by using a variety of different methods including use of sprays and gels. There are certain drawbacks to these manual processes including user error and lack of standardization. This can increase the risk that reusable instruments are not being properly disinfected and can result in harm to patients.

Accordingly, there is a need to automate and standardize a system and method for processing and cleaning reusable medical devices and instruments.

### SUMMARY

In concordance with the instant disclosure, an automated and standardized system and method for processing and cleaning reusable medical devices and instruments has surprisingly been discovered. The present technology includes articles of manufacture, systems, and processes that relate to a point-of-use system for the precleaning of medical instruments.

In certain embodiments, a system for cleaning a medical instrument is provided. The system can include a housing, a sanitizing drawer, a fluid reservoir, a spray nozzle, and a biohazard container. The sanitizing drawer can be disposed in the housing and can be configured to accept the medical instrument. The sanitizing drawer can open and close upon activation of a motion sensor disposed on the housing. The fluid reservoir can be disposed in the housing and can be configured to output a cleaning fluid to the sanitizing drawer. The spray nozzle can be disposed in the housing and can be configured to activate automatically after the sanitizing drawer is closed. The biohazard container can be removably disposed in the housing and can be positioned adjacent the sanitizing drawer. The biohazard container can be configured to accept the medical instrument from the sanitizing drawer automatically after the medical instrument is precleaned.

In certain embodiments, a method for precleaning a reusable medical instrument with a point-of-use cleaning system is provided. The method can include activating the motion sensor to open a drawer of the point-of-use system and placing the medical instruments in the sanitizing drawer. The motion sensor can again be activated to close the sanitizing drawer. The fluid nozzle can be actuated to spray the medical instruments and the medical instruments can be released from the sanitizing drawer into the biohazard container automatically.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a front perspective view of a point-of-use precleaning system for precleaning medical instruments, according to one embodiment;

FIG. 2 is a top, front perspective view of the system with a sanitizing drawer open for receiving medical instruments for precleaning, according to one embodiment;

FIG. 3 is a top, perspective, cutaway view of the sanitizing drawer of the system, including a spray nozzle and fluid reservoir, according to one embodiment;

FIG. 4 is a partial see-through front perspective view of the system with the spray nozzles activated, shown in phantom, according to one embodiment;

FIGS. 5A-5D are side, cutaway view of a biohazard container and a trap door of the system, according to one embodiment;

FIG. 6 is a front perspective view of the system with a housing door open to view a biohazard container, according to one embodiment;

FIG. 7 is a front perspective view of the system with the biohazard container removed, according to one embodiment;

FIG. 8 is a top-side perspective view of the fluid reservoir of the system, according to one embodiment;

FIG. 9 is a front elevational, partial cutaway view of the fluid reservoir and spray nozzles depicting a fluid path for cleaning fluid, according to one embodiment;

FIG. 10A is a front perspective view of a user interface of the system illustrating how access to the biohazard container can be granted, according to one embodiment;

FIG. 10B is a front perspective view of the user interface of the system illustrating the cleaning fluid level in the fluid reservoir, according to one embodiment;

FIG. 10C is a front perspective view of the user interface of the system illustrating how access to the fluid reservoir can be granted, according to one embodiment;

FIG. 10D is a front perspective view of the user interface of the system illustrating a fill level of the biohazard container, according to one embodiment;

FIG. 11 is a front perspective view of the system and various forms of methods to unlock and access the interior of the system, according to certain embodiments;

FIG. 12 is a system diagram depicting the point-of-use precleaning system, according to one embodiment; and

FIGS. 13A & 13B provide a flowchart depicting a method for precleaning a reusable medical instrument with a point-of-use cleaning system.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom.

Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves cleaning systems and methods for use in the outpatient and inpatient setting for precleaning medical devices and instruments after use but before sterilization. As used herein, a “medical instrument” can include various instruments used in a medical setting, such as one or more scalpels, forceps, scissors, speculums, dermatomes, biopsy punches, otoscope specula, drills, endoscopes, curettes, hemostats, bone chisels, retractors, needles, needle holders, laparoscopic instruments, and orthopedic pins and screws. A skilled artisan can select other such medical instruments within the scope of the present disclosure. The “medical instrument” can also include various reusable portions of medical devices.

As used herein, a “medical professional” can include any person in medicine who maintains health through the application of the principles and procedures of evidence-based medicine and caring. The “medical professional” can include various physicians, including specialists such as cardiologists, neurologists, and oncologists, surgeons, nurses, including registered nurses and nurse practitioners, physician assistants, pharmacists, dentists, and veterinarians.

It should also be appreciated that the present technology can be utilized across various medical disciplines, in both inpatient and outpatient settings, including surgery, dentistry, obstetrics, gynecology, ophthalmology, dermatology, gastroenterology, urology, otolaryngology, emergency medicine, podiatry, as well as veterinary medicine. A skilled artisan can select suitable areas of applicability within the scope of the present disclosure.

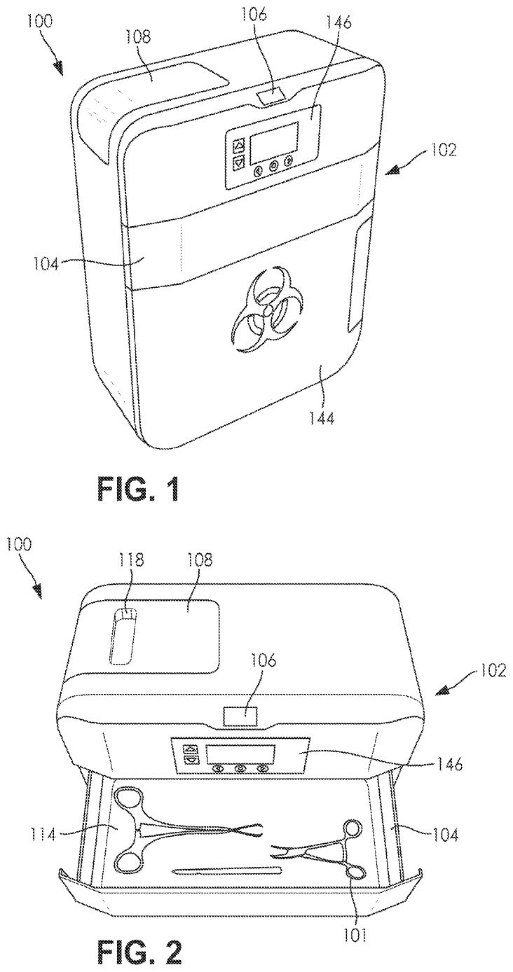

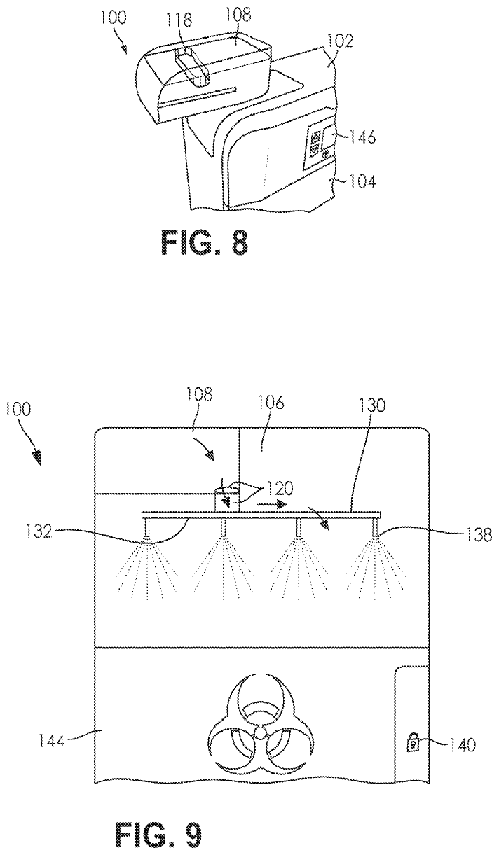

With reference now to the drawings, aspects of a point-of-use system 100 for precleaning medical instrument 101 are shown generally in FIGS. 1-12B. The system 100 can include a housing 102, a sanitizing drawer 104, a motion sensor 106, a fluid reservoir 108, a spray nozzle 110, and a biohazard container 112. The sanitizing drawer 104 can be disposed in the housing 102 and can be configured to accept the medical instrument 101. The sanitizing drawer 104 can be configured to open and close upon activation, by a medical professional, of the motion sensor 106 disposed on the housing 102. The fluid reservoir 108 can be disposed in the housing 102 and can be configured to output a cleaning fluid 103 to the sanitizing drawer 104. The spray nozzle 110 can be disposed in the housing 102 and can be configured to activate automatically after the sanitizing drawer 104 is closed. The biohazard container 112 can be removably disposed in the housing 102 and can be positioned adjacent the sanitizing drawer 104. The biohazard container 112 can be configured to accept the medical instrument 101 from the sanitizing drawer 104 automatically after the medical instrument 101 is precleaned.

With reference to FIGS. 1, 3, and 6, the housing 102 of the system 100 can accommodate several components, including the sanitizing drawer 104, the fluid reservoir 108, the spray nozzle 110, and the biohazard container 112. The housing 102 can be constructed in various sizes and shapes to suit different medical environments, ranging from compact models for small clinics to larger units for hospital settings, as examples. A skilled artisan can select a suitable size and shape for the housing 102 within the scope of the present disclosure. The housing 102 can be constructed of a rigid, durable material that is resistant to corrosion, such as stainless steel or high-grade plastics, which are suitable for maintaining sterility and withstanding frequent cleaning and disinfection processes. As a non-liming example, the housing 102 can be formed of stainless steel, anodized aluminum, polycarbonate, acrylic, polypropylene, acrylonitrile butadiene styrene, or fiberglass. A skilled artisan can select a suitable material for forming the housing 102 within the scope of the present disclosure.

Within the housing 102, the sanitizing drawer 104 can be placed to allow for easy access for loading and unloading medical instruments 101. The fluid reservoir 108 can be disposed adjacent to the sanitizing drawer 104 and can be configured to supply the necessary cleaning fluid 103 directly to the spray nozzles 110. The spray nozzles 110 can activate automatically and deliver a thorough precleaning spray to the medical instrument 101 once the drawer is closed. Additionally, the biohazard container 112 can be removably disposed within the housing 102 and can be positioned to receive the medical instrument 101 directly from the sanitizing drawer 104 after the precleaning process, facilitating safe and efficient disposal or further processing of the precleaned medical instrument 101.

As shown in FIG. 1, an exterior of the housing 102 includes the motion sensor 106, which can facilitate the usability and hygiene of the system 100. The motion sensor 106 can allow for touchless operation of the sanitizing drawer 104, militating against cross-contamination and providing convenience to the user. By waving a hand near the motion sensor 106, the medical professional can initiate the opening or closing of the sanitizing drawer 104, allowing the system 100 to operate with hands free interaction facilitating cleanliness in environments where sterility and quick operation are paramount.

The housing 102 can be both airtight and liquid-tight to militate against chemicals escaping during the precleaning process. This containment can assist with maintaining a working environment free from hazardous chemicals, particularly in medical settings where exposure to potent cleaning chemicals can pose health risks. The airtight and liquid-tight seals around the housing 102 militate against the volatilization of chemical vapors and the leakage of fluids, respectively. The seals of the housing 102 can be made from robust materials that offer resistance to the corrosive nature of various cleaning fluid 103s and withstand the pressure and temperature changes that occur during the precleaning cycle. By containing these substances, the system 100 also helps in maintaining the integrity of the cleaning process, ensuring that the chemical concentration remains consistent and effective throughout the cleaning operation. Furthermore, this minimizes the environmental impact of the system 100 by reducing the emission of volatile organic compounds into the atmosphere.

The sanitizing drawer 104 can facilitate the initial precleaning and sanitization process immediately after use. The sanitizing drawer 104 can accommodate a variety of medical instruments 101, providing a secure and controlled environment for the preliminary removal of biological contaminants such as blood, tissue, and other organic matter. The sanitizing drawer 104 can be constructed from materials like stainless steel or high-grade plastics, which are chosen for their durability and ease of cleaning, as well as their ability to withstand repeated exposure to harsh cleaning fluid 103. A skilled artisan can select a suitable material for the sanitizing drawer 104 within the scope of the present disclosure.

Functionally, the sanitizing drawer 104 can operate with automation to enhance efficiency. The sanitizing drawer 104 can be in communication with the motion sensor 106 that allows for touchless operation, thereby militating against cross-contamination. Upon activation of the motion sensor 106, the sanitizing drawer 104 can open, as shown in FIG. 2, to receive the used medical instrument 101. After the medical instrument 101 is placed inside the sanitizing drawer 104, the motion or an additional command can be used to close the sanitizing drawer 104, which can activate the precleaning process. Inside, the strategically placed spray nozzle 110 deliver a precise amount of cleaning fluid 103, which is stored in the fluid reservoir 108 within the housing 102. This can facilitate the medical instrument 101 being uniformly exposed to the cleaning fluid 103, effectively addressing the initial bioburden and preparing the medical instruments 101 for further sterilization processes.

The sanitizing drawer 104 can enhance user convenience and promote hygiene with additional automated actions. Once the precleaning cycle is complete, the medical instrument 101 can be automatically transferred to the biohazard container 112 for further decontamination and additional processing. This transition between the sanitizing drawer 104 and the biohazard container 112 can not only save time but also significantly militate against the handling of potentially contaminated medical instruments 101, thereby promoting a safe medical environment.

It should be appreciated that the sanitizing drawer 104 can include additional sensors to optimize the precleaning process and promote thorough sanitization of medical instruments 101. The sensors can include a weight sensor 113 for detecting the presence of a medical instrument 101 and estimate the load of the medical instruments 101 placed within the sanitizing drawer 104, aiding in the adjustment of the cleaning fluid 103 volume and spray intensity. The sanitizing drawer 104 can further include a temperature sensor, a light sensor, a humidity sensor, and a proximity sensor, as examples.

The sanitizing drawer 104 can be liquid-tight, militating against cleaning fluid 103 from leaking or spilling from the sanitizing drawer 104, in embodiments where the sanitizing drawer 104 is filled to a certain extent with cleaning fluid 103. This feature can allow for effective soaking or immersion precleaning processes where the medical instrument 101 requires thorough saturation with enzymatic or detergent-based solutions to loosen and remove biological contaminants before further sterilization. The liquid-tight construction of the sanitizing drawer 104 can involve precision seals and gaskets along its edges and at the connection points, which militate against fluid escaping even when the sanitizing drawer 104 is filled to capacity. Additionally, the material used for the sanitizing drawer 104 can be selected for chemical resistance and durability, ensuring that the material does not degrade or warp in the presence of various cleaning fluids 103.

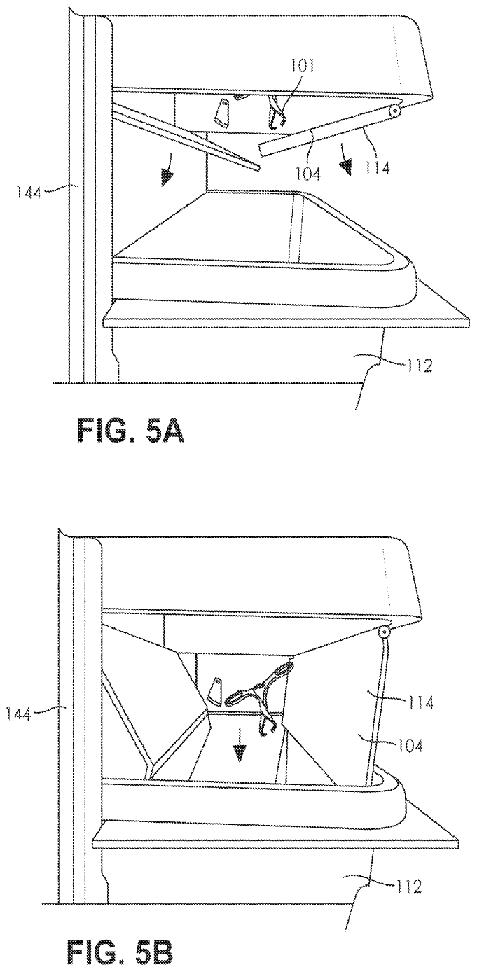

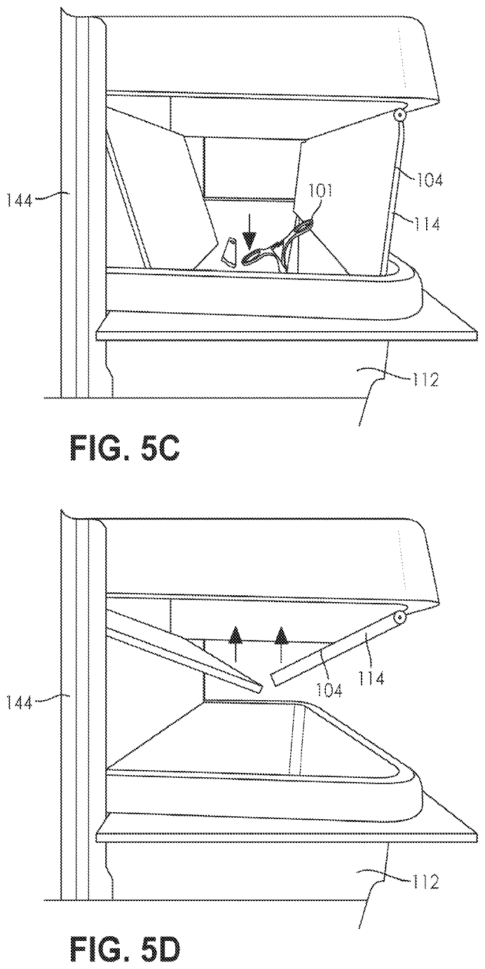

With reference to FIGS. 2 and 5A-5D, the sanitizing drawer 104 can include a false bottom 114 to facilitate the transfer of a precleaned medical instrument 101 into the biohazard container 112 for further processing. The false bottom 114 can be configured to promote safe and efficient handling of delicate medical instruments 101. In one embodiment, the false bottom 114 can include hinged, overlapping trap doors. These doors can open sequentially or simultaneously to create an opening through which the medical instrument 101 can gently drop into the biohazard container 112 positioned directly below the sanitizing drawer 104. The trap doors can be precisely controlled by a controller 116 synchronized with the precleaning cycle, such that the doors only open once the precleaning process is complete and the medical instrument 101 is ready for the next stage of sterilization.

In an alternative embodiment, the false bottom 114 can be lowered into the biohazard container 112. The bottom of the sanitizing drawer 104 can descend, bringing the medical instrument 101 into to the biohazard container 112, thereby minimizing the distance the medical instrument 101 falls between the sanitizing drawer 104 and the biohazard container 112 when released. Advantageously, this feature can be useful when handling a delicate or finely calibrated medical instruments 101 that could be damaged by even a short drop. By lowering the medical instrument 101 gently into the biohazard container 112, the risk of impact damage can be minimized.

In yet another embodiment, the false bottom 114 can angle downward, creating a slide (not shown) that guides the medical instrument 101 into the biohazard container 112. The slide can be advantageous as it combines the benefits of controlled movement with gravity-assisted transfer, allowing the medical instrument 101 to glide smoothly into the biohazard container 112 without significant impact. The slide can also speed up the process of transferring medical instruments 101, enhancing overall efficiency in busy medical settings.

With reference to FIGS. 1, 3 and 8-9, the fluid reservoir 108 can be integrated within the housing 102 of the system 100 to allow for a seamless and efficient flow of cleaning fluid 103 to the spray nozzle 110. With reference to FIG. 9, the fluid path of the cleaning fluid through the system 100 is shown. The fluid reservoir 108 can be easily accessible for refilling and maintenance. In some embodiments, the fluid reservoir 108 can include a handle 118 or other ergonomic element that facilitates quick and easy handling by the medical professional. The fluid reservoir 108 can accommodate various types of cleaning fluids 103, allowing it to be adaptable to different cleaning protocols required by specific medical instruments 101. Additionally, the fluid reservoir 108 can include a level indicator and/or a sensor that is in communication with the controller 116 and is configured to alert the medical professional when the fluid level is low, ensuring that the system 100 is prepared for operation without interruption. The fluid reservoir 108 can include a spout 120, which is dimensioned to fit into an aperture 122 on the system 100. In this way, the medical professional can change out the fluid in the fluid reservoir 108 after the precleaning is complete.

It should be appreciated that the cleaning fluid 103 can include various fluids and hydrogels for the cleaning and pre-cleaning of medical instruments 101, each tailored to effectively remove biological contaminants and prepare medical instruments 101 for sterilization. Enzymatic cleaners are widely used due to their ability to break down proteins, fats, and other organic materials, making them particularly effective against blood and tissue residues. Alkaline detergents are another common choice, appreciated for their capacity to dissolve fats and oils while also providing excellent cleaning in hard water conditions. For medical instruments 101 contaminated with more stubborn residues, acidic cleaners can be utilized to remove mineral deposits and other inorganic matter. Additionally, alcohol-based solutions might be used for their rapid microbial action and quick drying properties, although they are generally employed as a final rinse or in situations requiring quick turnaround. Each of these cleaning fluids 103, and others selected by a skilled artisan, can be used in the fluid reservoir 108, depending on the specific requirements of the medical facility and the nature of the contaminants present on the instruments 101.

Referring now to FIG. 3, a top-front perspective view of a drawer of the system 100 for pre-cleaning medical instruments 101 is shown. The internal components can include a motor or actuator 124, a fluid inlet 126, a fluid pipe 128, a first spray bar 130 having multiple spray nozzles 134 and a second spray bar 132 having multiple spray nozzles 138. In operation, when the medical professional closes the drawer 104, the motor 124 can be configured to actuate and pull cleaning fluid 103 from the fluid reservoir 108 via fluid pipe 128 and fluid inlet 126. Cleaning fluid 103 is then configured to flow to the first spray bar 130 and the second spray bar 132, where it is dispensed from the multiple spray nozzles 134, 138.

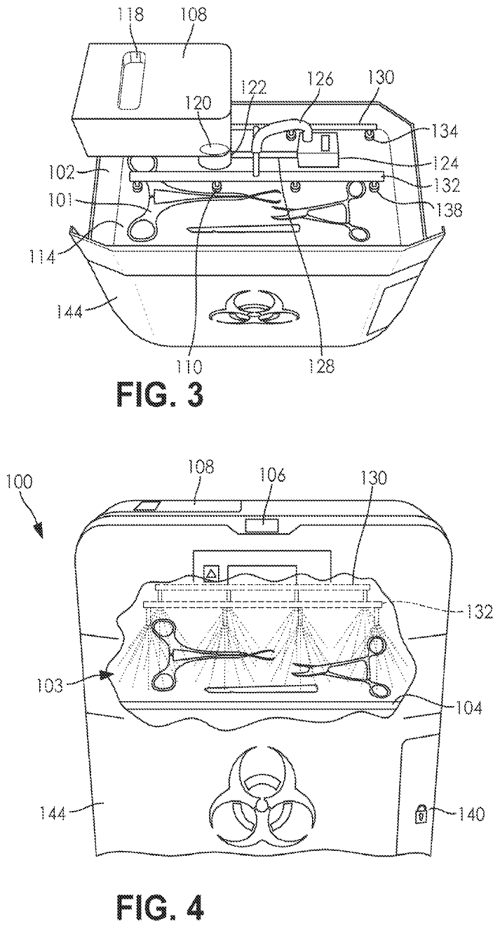

Referring now to FIG. 4, a partial see-through front perspective view of the system 100 for pre-cleaning medical instruments 101 is shown. The spray nozzles 110 can be actuated by the motor 124 and cleaning fluid 103 can be dispensed from the spray nozzles 134, 138 to preclean the medical instruments 101 in the sanitizing drawer 104 with the enzymatic fluid from the fluid reservoir 108. The fully contained sanitizing drawer 104 with the medical instrument 101, can be sprayed at a downward angle by the spray nozzles 134, 138. Additionally, the spray nozzles 134, 138 can move and rotate on the spray bars 130, 132 to allow for varying multi-angle spray capabilities. This can include top and bottom, simultaneous sprays or side sprays. As described herein, the sanitizing drawer 104 can include internal sensors to turn on sprayers, based on the quantity of medical instruments 101 in the tray, as well as the area in the sanitizing drawer 104 where the medical instruments 101 were placed. In operation, this ensures that an optimized amount of cleaning fluid 103 is sprayed over the medical instruments 101. In certain embodiments, the sanitizing drawer 104 can fill up with the cleaning fluid 103 to ensure that difficult to clean medical instruments 101 receive proper fluid treatment on their surfaces.

In certain embodiments, the false bottom 114 of the sanitizing drawer 104 can include spray nozzles 110 configured to spray the bottom of the medical instrument 101 with the cleaning fluid 103. The spray nozzles 110 can be positioned at various internals along the false bottom 114 to facilitate even distribution of the cleaning fluid 103. Further, the spray nozzles 103 can pressurize the cleaning fluid 103 to allow for the cleaning fluid 103 to be sprayed upward with enough pressure to adequately preclean the medical instrument 101 and remove or dislodge biological material, such as blood and tissue, from the medical instrument 101.

It should be appreciated that a diagnostic sensor 135 can be integrated within the system 100 to monitor, and in certain instances, change, operational parameters such as temperature, humidity, ultraviolet (UV) light, and spray nozzle functionality, where any deviation from one or more predefined thresholds can trigger an immediate diagnostic check. The diagnostic sensors can conduct self-tests at regular intervals and report the health status of the system components to a maintenance dashboard accessible by authorized personnel.

The system 100 and sanitizing drawer 104 can further include a UV sensor in communication with the controller 116. The UV sensor can detect the presence and intensity of UV radiation, which can be used for further sterilization and disinfection of the medical instruments 101. By monitoring the UV light levels, the UV sensor can determine that the UV radiation is within the required thresholds to effectively eliminate microbial contaminants without damaging the medical instrument 101. This feedback can allow for real-time adjustments by the system 100 or the medical professional. The system 100 can also include a temperature sensor in communication with a heating element that serves a dual function in maintaining and regulating the temperature within the system 100. By controlling temperature in this way, the system 100 can further sanitize using temperature. The temperature sensor can continuously monitor the temperature of the sanitizing drawer 104 or a specific component, such as the fluid reservoir 108. This data can be communicated to the heating element, which can adjust output to maintain the desired temperature setpoint. In other embodiments, the system 100 can include a humidity sensor for measuring the moisture content in the air within a system 100. The humidity sensor can monitor the humidity levels in the housing 102 to be controlled to militate against the growth of mold and bacteria, or to promote optimal conditions for certain chemical processes or storage. In the context of the present system 100, controlling humidity can militate against the premature drying of cleaning fluid 103 on the medical instrument 101 or to maintain the integrity of moisture-sensitive components and surfaces. Each of the sensors discussed hereinabove be controlled by the controller 116 in communication with the various sensors employed.

The sanitizing drawer 104 can further include a stabilization mechanism to keep the medical instrument 101, such as scissors or forceps, in an open configuration during the precleaning process. Optionally, this can be achieved through the use of adjustable holders or clips within the sanitizing drawer 104 that can securely grip the medical instrument 101 in an open position, ensuring that all surfaces are exposed to the cleaning fluid 103s. Additionally, the system 100 can utilize a combination of spraying and a ‘jiggle’ technique, where the sanitizing drawer 104 gently agitates the medical instrument 101 to help dislodge and remove any tissue or debris caught in the medical instrument 101 mechanisms. Despite this movement, the stabilization features can be robust enough to maintain the medical instrument 101 in the open configuration throughout the process. This can aid with a thorough cleaning by allowing the cleaning fluid 103, via the spray nozzle 110, to adequately reach all parts of the medical instrument 101, enhancing the effectiveness of the sanitization cycle.

In certain embodiments, the sanitizing drawer 104 can accommodate an entire surgical tray of medical instruments 101 as opposed to individual medical instruments 101. In such embodiments, the medical instruments 101 are able to transition into a separate biohazard container 112 that envelopes the entire tray of medical instruments 101. This allows for a more accurate count of all the medical instruments 101 and pieces at the conclusion of a medical procedure, as well as providing a means to transport the set of related instruments back to sterile processing for the next steps. In this embodiment, the system 100 can complete the spray cycle and then release the tray out of the sanitizing drawer 104 so that it can be taken back into sterile processing in the same surgical tray, rather than falling into the biohazard container 112.

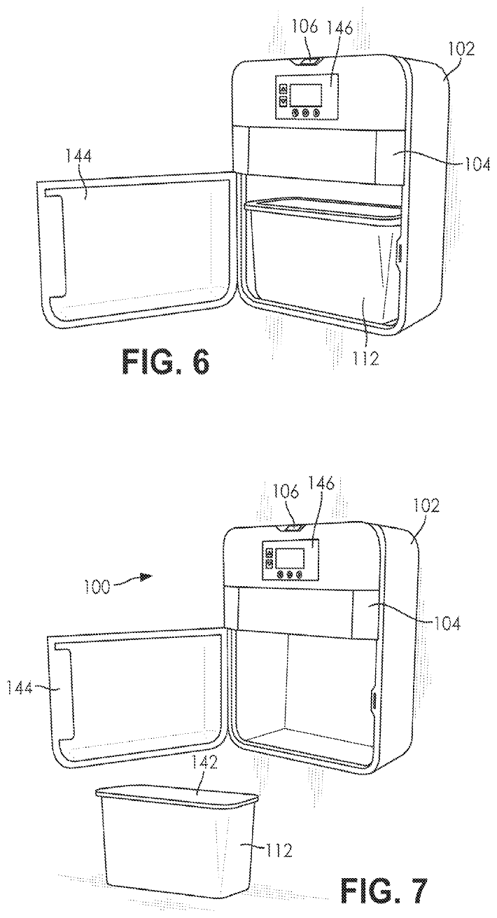

With reference to FIG. 6, the biohazard container 112 can be sized to fit within the housing 102 and can be positioned directly beneath the sanitizing drawer 104 to allow for the medical instrument 101 to be automatically dropped into the biohazard container 112 once the precleaning cycle is complete. The biohazard container 112 can be constructed from materials such as high-density polyethylene (HDPE) or stainless steel, which are known for their durability and resistance to corrosion from chemical cleaners. A skilled artisan can select other suitable materials. Additionally, the biohazard container 112 can be configured to be coupled with a lid 142, shown in FIG. 7, which aids in safely containing the biohazardous materials and prevents spillage during transport for further processing.

The biohazard container 112 can also be filled with a secondary cleaning fluid 103, providing an additional stage of cleaning or disinfection as needed and can keep the medical instruments 101 wet in between the precleaning process carried out by the system 100 and the next step in the sterilization process. Advantageously, keeping the medical instrument 101 wet in this way can militate against tissue and other debris drying on the medical instrument 101 which can result in the medical instrument 101 being unable to be fully cleaned, which may require the medical instrument 101 to be discarded. The secondary cleaning fluid 103 can serve various purposes, such as neutralizing any remaining contaminants or preparing the medical instruments 101 for further sterilization steps.

Referring now to FIGS. 1 and 7, a front-side perspective view the system 100 is shown. The housing 102 can include a door 144 which can be configured to open to allow access to the biohazard container 112. As shown in FIG. 6, the door 144 can allow access to only the biohazard container 112 to militate against a medical instrument 101 that is currently in the sanitizing drawer 104 from contaminating the open air. The housing 102 can also include a user interface 146, which can allow for a registered user of the system 100, such as a medical professional, to scan their ID and open the housing 102 and access the biohazard container 112. The medical professional can retrieve the biohazard container 112 from the housing 102, place a lid 142 on the biohazard container 112, as shown in FIG. 7, and the biohazard container 112 can be transported to the next step of the sterilization process.

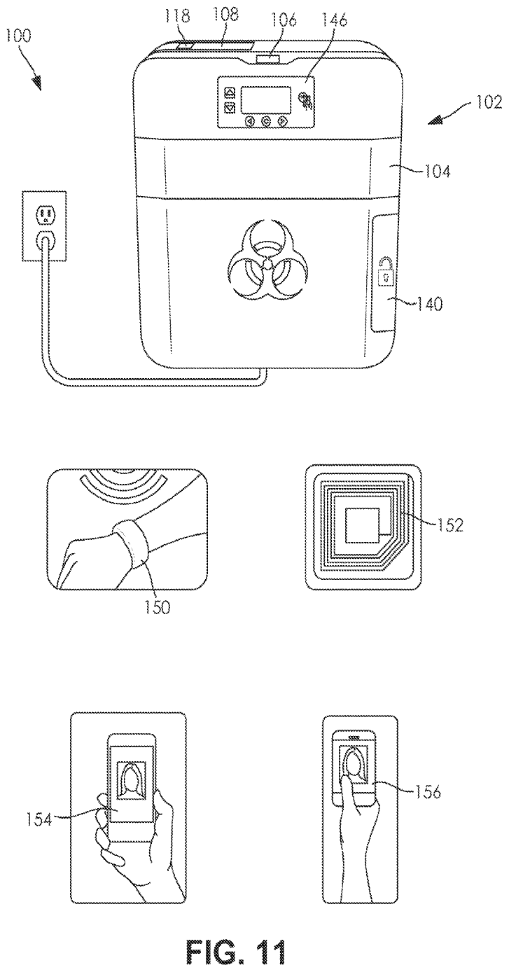

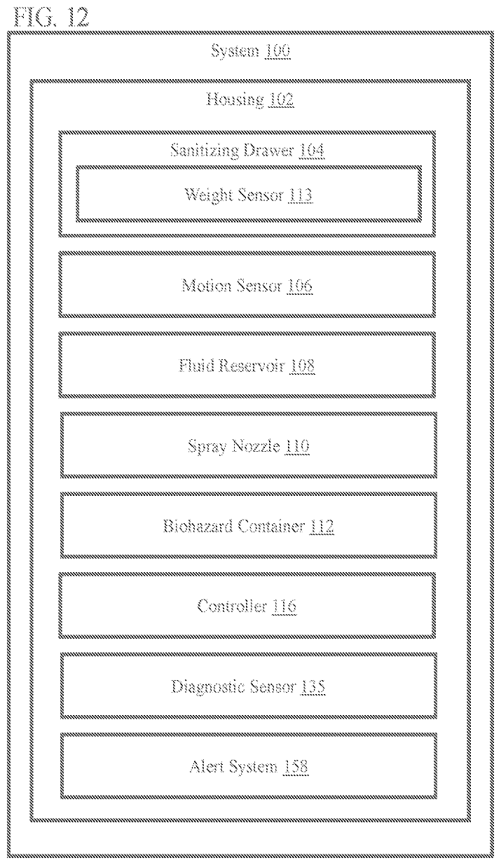

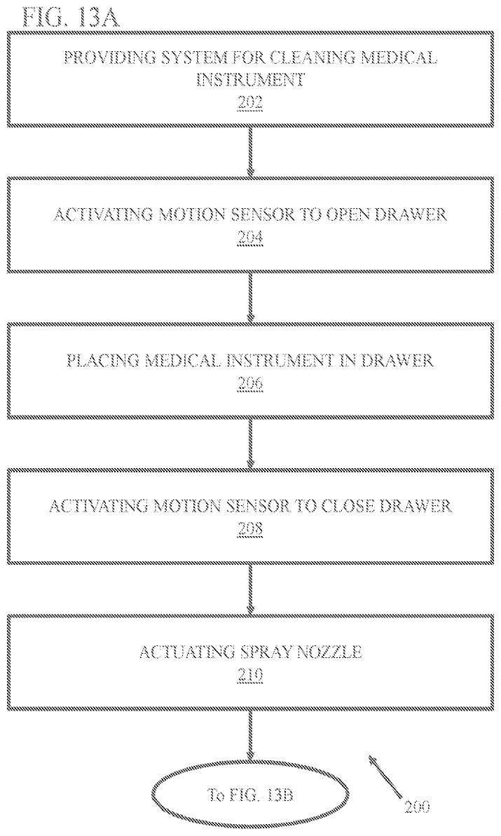

The system 100 can include a door locking mechanism 140, as shown in FIG. 4, that can be either manual, utilizing a key, or electronic, controlled by the controller 116 and interfaced through the user interface 146. The locking mechanism 140 can secure the biohazard container and the sanitizing drawer 104. With respect to the sanitizing drawer 104, the locking mechanism 140 can engage automatically when the sanitizing drawer is closed by the medical professional. This dual approach allows for flexibility in security measures according to the needs of the medical facility. In the electronic embodiment, the user interface 146 can be programmed to require an ID scan from a registered user, such as a medical professional, to grant access to the biohazard container 112. This feature ensures that only authorized personnel can handle the potentially hazardous contents. The integration of an electronic lock managed by the controller 116 can enhance the security protocols of the system 100, making it a reliable component in the handling and disposal of medical instruments 101. Referring to FIG. 11, various ways to unlock and access the system 100 are shown, including a radio frequency (RF) ID bracelet 150, an RF ID sticker FOB 152, a phone near field communication 154, or a badge RF ID 156.

Referring now to FIG. 1, which illustrates a front perspective view of the user interface 146 of the system 100, the user interface 146 can display essential information such as the status of the precleaning cycle, fluid levels in the fluid reservoir 108, and the condition of the biohazard container 112. This real-time feedback allows medical professionals to be informed about the operation of the system 100 and can make adjustments as necessary. Additionally, the user interface 146 can incorporate touch-sensitive screens or buttons that are easy to clean and disinfect, to further aid in maintaining a sterile environment. The interface can also be customized to include language options and symbols that are universally understood, making the system 100 accessible to a diverse workforce. In a preferred embodiment, the user interface 146 can be in communication with the motion sensor 106 to allow for the medical professional to make motions with their hand to user the user interface 146 as well as make selections on the user interface 146.

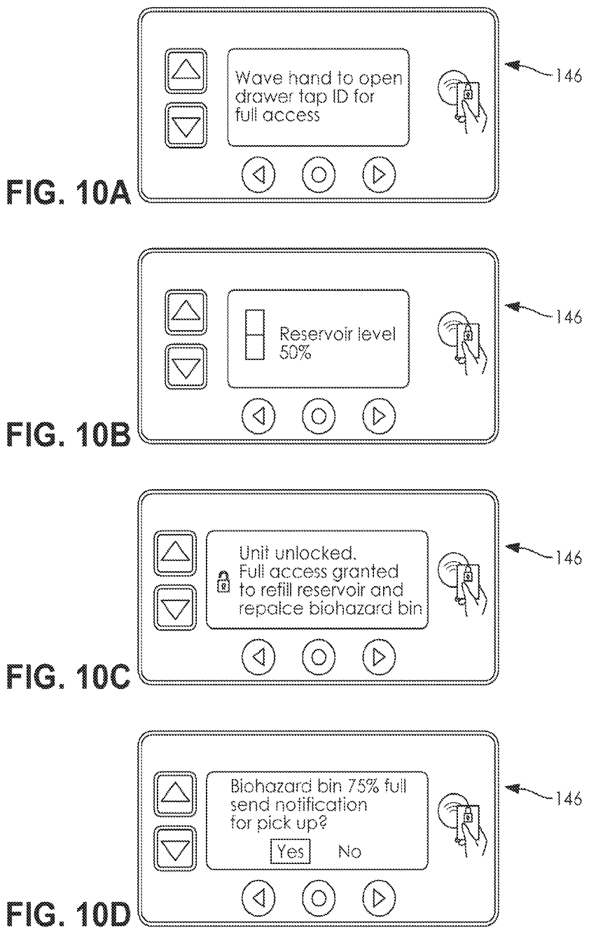

With reference to FIGS. 10A-10D, the user interface 146 is shown in four exemplary configurations. At FIG. 10A, the user interface 146 shows a configuration in which the user is able to access the drawer. In operation, the user can waive their hand in front of the motion sensor 106. The medical professional can have a credentialed badge with certain levels of securitization for access to provide security in operation of the system 100. At FIG. 10B, the user interface 146 shows the fluid reservoir 108 level. At FIG. 10C, the user interface 146 shows the fluid reservoir 108 needs to be refilled and the biohazard container 112 needs to be emptied or replaced. At FIG. 10D, the biohazard container 112 amount is shown on the user interface 146. To further assist the medical professional in maintaining the system 100, a notification can be sent to the medical professional via the controller 116 to alert the medical professional that the biohazard container 112 is full and needs replaced. In certain embodiments, the controller 116 can be configured to send notifications to a central monitoring system regarding the status of the precleaning process, the diagnostic sensor data, the cleaning fluid 103 level, the biohazard container 112 fill level, and other data as selected by a skilled artisan. In certain embodiments, the system 100 can display on the user interface 146 or notify the central monitoring system of system 100 malfunctions, or unauthorized access attempts, enhancing the overall security and reliability of the system 100.

In certain embodiments, the user interface 146 can include security features. The security features can require authentication, such as ID scans, passwords, or biometric data, such as facial recognition or fingerprints, to access sensitive components of the system 100 like the biohazard container 112. These security measures can ensure that access is restricted to authorized personnel only, safeguarding against unauthorized use. In other embodiments, the system 100 can include an emergency stop feature that allows medical professionals to immediately halt the cleaning process in case of an emergency, enhancing user control.

The system 100 can be communicatively coupled to one or more remote platforms. The communicative coupling can include communicative coupling through a networked environment. The networked environment can be a radio access network, such as LTE or 5G, a local area network (LAN), a wide area network (WAN) such as the Internet, or wireless LAN (WLAN), for example. Implementations also include where the system 100 can be operatively linked via some other communication coupling to one or more computing platforms and remote platforms. The one or more one or more computing platforms can be configured to communicate with the networked environment via wireless or wired connections. In addition, in an embodiment, the one or more computing platforms can be configured to communicate directly with each other via wireless or wired connections. Examples of one or more computing platforms can include smartphones, wearable devices, tablets, laptop computers, desktop computers, Internet of Things (IoT) device, or other mobile or stationary devices. In certain embodiments, a system can be provided that can also include one or more hosts or servers, such as the one or more remote platforms connected to the networked environment through wireless or wired connections. Remote platforms can be implemented in or function as base stations (which can also be referred to as Node Bs or evolved Node Bs (eNBs)). In certain embodiments, remote platforms can include web servers, mail servers, application servers, etc. According to certain embodiments, remote platforms can be standalone servers, networked servers, or an array of servers.

The system 100 can include one or more processors for processing information and executing instructions or operations, including such instructions and/or operations stored on one or more non-transitory mediums. One or more processors can be any type of general or specific purpose processor. In some cases, multiple processors can be utilized according to other embodiments. In fact, the one or more processors can include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In some cases, the one or more processors can be remote from the one or more computing platforms. The one or more processors can perform functions associated with the operation of the system 100 which can include, for example, precoding of antenna gain/phase parameters, encoding and decoding of individual bits forming a communication message, formatting of information, and overall control of the one or more computing platforms, including processes related to management of communication resources.

The system 100 can further include or be coupled to a memory (internal or external), which can be coupled to one or more processors, for storing information and instructions that can be executed by one or more processors, including any instructions and/or operations stored on one or more non-transitory mediums. Memory can be one or more memories and of any type suitable to the local application environment and can be implemented using any suitable volatile or nonvolatile data storage technology such as a semiconductor-based memory device, a magnetic memory device and system, an optical memory device and system, fixed memory, and removable memory. For example, memory can consist of any combination of random access memory (RAM), read only memory (ROM), static storage such as a magnetic or optical disk, hard disk drive (HDD), or any other type of non-transitory machine or computer readable media. The instructions stored in memory can include program instructions or computer program code that, when executed by one or more processors, enable the one or more computing platforms to perform tasks as described herein.

In some embodiments, one or more computing platforms can also include or be coupled to one or more antennas for transmitting and receiving signals and/or data to and from one or more computing platforms. The one or more antennas can be configured to communicate via, for example, a plurality of radio interfaces that can be coupled to the one or more antennas. The radio interfaces can correspond to a plurality of radio access technologies including one or more of LTE, 5G, WLAN, Bluetooth, near field communication (NFC), radio frequency identifier (RFID), ultrawideband (UWB), and the like. The radio interface can include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

It should be appreciated that the controller 116 can control several operational features of the system 100. For example, the controller 116 can the open and close the sanitizing drawer 104, which can assist with maintaining a sterile environment and preventing unauthorized access during the precleaning cycle. The controller 116 can regulate the activation of the spray nozzle 110, ensuring that the correct amount of cleaning fluid 103 is dispensed and that the timing of the spray aligns perfectly with the precleaning protocol. This precise control helps in achieving consistent and effective cleaning results. Furthermore, the controller 116 can manage the fluid dynamics within the system 100, controlling the fluid reservoir 108 to ensure that there is always an adequate supply of cleaning fluid 103 available. The controller 116 can monitor the levels and can alert the medical professional via the user interface 146 when the fluid needs to be replenished. This feature helps in maintaining the efficiency of the system 100 and militates against any interruptions in the precleaning process.

The system 100 can include an alert system 158 configured to notify the medical professional of at least one of a low fluid level of the fluid reservoir 108, a system 100 malfunction, an unauthorized access attempt to the system 100, and whether the system 100 itself requires cleaning. The alert system 158 can alert the medical professional through the user interface 146. Advantageously, the alert system 158 can militate against the medical professional frequently checking the system 100. A skilled artisan can select other such features for the alert system 158 to notify the medical professional about within the scope of the present disclosure.

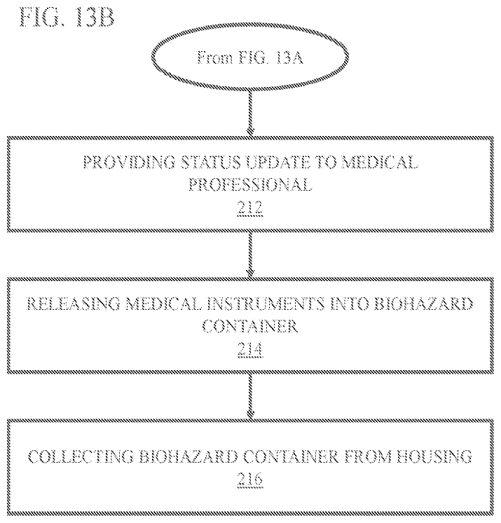

The present disclosure further provides a method 200 for pre-cleaning a reusable medical instrument 101 with a cleaning system 100, as shown in FIGS. 13A-13B. The method 200 can include a step 202 of providing the system 100 of the present disclosure. In a step 204, the method 200 can include activating the motion sensor 106 to open the sanitizing drawer 104 of the system 100. The medical professional can place the medical instruments 101 in the sanitizing drawer 104 in a step 206 and activate the motion sensor 106 to close the sanitizing drawer 104 in a step 208. The method 200 can include a step 210 of actuating, by the system 100, the spray nozzles 110 to spray the medical instruments 101. In a step 212, the system 100 can provide the medical professional with a status update via the user interface 146 during the precleaning process. The system 100 can release the medical instruments 101 from the sanitizing drawer 104 into the biohazard container 112 automatically in a step 214. In a step 216, the medical professional can collect the biohazard container 112 from the housing 102 to allow for the medical instruments 101 to continue in the sterilization process.

Advantageously, the system 100 and method 200 described herein automate the precleaning process thereby eliminating user errors, providing standardization, and militating against hazardous chemical exposure by medical professionals and patients.

### EXAMPLES

### Example 1: Emergency Room Scenario

In a hospital emergency room, medical professionals frequently use a variety of instruments that must be quickly and effectively precleaned to ensure that residual tissue does not become adhered to the medical instruments rendering them unusable and requiring them to be permanently disposed. The system can be installed directly in the emergency room to facilitate immediate pre-cleaning of instruments such as scalpels, forceps, and scissors right after their use.

After treating a patient with a deep laceration, the used surgical instruments are contaminated with blood and other biological materials. Instead of sending these instruments directly to the central sterilization department, which can allow blood and biological materials to dry onto the surgical instruments, the attending nurse uses the precleaning system.

A nurse activates the system by waving a hand over the motion sensor, opening the sanitizing drawer. The contaminated instruments are placed inside the drawer. The drawer is closed using the same touchless gesture, and the automated precleaning cycle begins, spraying the instruments with an enzymatic cleaning solution. Once the precycle is complete, the surgical instruments are automatically dropped into a biohazard container for safe storage until they can be further sterilized. This immediate pre-cleaning helps to militate against the drying of biological materials on the instruments, making them easier to fully sterilize later.

### Example 2: Outpatient Surgical Center

Outpatient surgical centers perform numerous minor surgeries and procedures daily, requiring a fast turnaround of patient rooms. The system provides a rapid and standardized method of precleaning, which can be important for maintaining the workflow. During a day filled with back-to-back minor orthopedic surgeries, various instruments are used repeatedly. To maintain efficiency, these instruments need to be quickly precleaned and prepared for sterilization. After a procedure, the surgical technician activates the precleaning system by a touchless gesture, opening the sanitizing drawer.

The medical instruments used during the surgery are placed in the drawer, which is then closed using the motion sensor. The system automatically initiates the precleaning cycle, applying a high-efficacy enzymatic spray to the medical instruments. After precleaning, the medical instruments are automatically released into a biohazard container, ready to be moved to the sterilization department. This system not only saves time but also enhances the overall efficiency of the surgical center by allowing staff to handle medical instrument cleaning within the procedure area, thus speeding up the preparation for subsequent surgeries.

These examples illustrate how the precleaning system can be integrated into different medical settings to improve the efficiency of instrument reprocessing and ensure compliance with hygiene standards.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A point-of-use system for precleaning a medical instrument via a precleaning process, the system comprising:
a housing;
a sanitizing drawer disposed in the housing and configured to accept the medical instrument and to open and close upon activation of a motion sensor disposed on the housing;
a fluid reservoir disposed in the housing and configured to hold a cleaning fluid and output the cleaning fluid to the sanitizing drawer, the fluid reservoir fluidly coupled to the spray nozzle;
a spray nozzle disposed in the housing and configured to activate automatically after the sanitizing drawer is closed, the spray nozzle fluidly coupled to the fluid reservoir; and
a biohazard container removably disposed in the housing and positioned adjacent the sanitizing drawer, the biohazard container configured to accept the medical instrument from the sanitizing drawer automatically after the medical instrument is precleaned.

2. The system of claim 1, further comprising a user interface configured to display a status of the precleaning process.

3. The system of claim 2, wherein the user interface includes a biometric security feature configured to allow access to the sanitizing drawer based on fingerprint recognition or facial recognition.

4. The system of claim 1, wherein the fluid reservoir includes a handle.

5. The system of claim 1, wherein the spray nozzle is comprised by a plurality of spray nozzles positioned to provide coverage of the medical instrument.

6. The system of claim 5, further including a motor configured to transfer the cleaning fluid from the fluid reservoir to the spray nozzle.

7. The system of claim 6, further including a fluid pipe and fluid inlet configured to move cleaning fluid from the fluid reservoir to the spray nozzle and in fluid communication with the fluid reservoir and the spray nozzles.

8. The system of claim 1, wherein the sanitizing drawer includes a trap door configured to release the medical instrument into the biohazard container.

9. The system of claim 8, wherein the trap door is configured to automatically actuate after the precleaning process is complete.

10. The system of claim 1, wherein the cleaning fluid comprises an enzymatic solution configured to break down organic matter.

11. The system of claim 1, wherein the sanitizing drawer includes a weight sensor for detecting a presence and position of medical instruments within the sanitizing drawer.

12. The system of claim 1, further including a controller configured to communicate with a user interface.

13. The system of claim 12, wherein the data includes a member selected from a group consisting of an operation of the precleaning process, a fluid level, a system alert, and combinations thereof.

14. The system of claim 1, further comprising a locking mechanism to secure the biohazard container and the sanitizing drawer.

15. The system of claim 14, wherein the locking mechanism is configured to engage automatically when the sanitizing drawer is closed.

16. The system of claim 1, further including a diagnostic sensor configured to monitor an operational parameter including a member selected from a group consisting of temperature, fluid pressure, spray nozzle functionality, and combinations thereof.

17. The system of claim 1, further including an alert system configured to notify a medical professional of at least one of a low fluid level, a system malfunction, and an unauthorized access attempt.

18. A point-of-use system for precleaning a medical instrument via a precleaning process, the system comprising:
a housing having a locking mechanism;
a sanitizing drawer disposed in the housing and configured to accept the medical instrument and to open and close upon activation of a motion sensor disposed on the housing, the sanitizing drawer including
a trap door on a bottom of the sanitizing drawer, and
a sensor for detecting a presence and position of medical instruments within the sanitizing drawer;
a fluid reservoir disposed in the housing and configured to hold a cleaning fluid and output the cleaning fluid to the sanitizing drawer, the fluid reservoir fluidly coupled to the spray nozzle, the fluid reservoir including a handle;
a spray nozzle disposed in the housing and configured to activate automatically after the sanitizing drawer is closed, the spray nozzle fluidly coupled to the fluid reservoir, the spray nozzle having a plurality of spray nozzles positioned to provide multi-angle coverage of the medical instrument;
a biohazard container removably disposed in the housing and positioned adjacent the sanitizing drawer, the biohazard container configured to accept the medical instrument from the sanitizing drawer automatically after the medical instrument is precleaned;
a user interface disposed on the housing and configured to display a status of the precleaning process;
a motor configured to actuate and pull fluid from the fluid reservoir to the spray nozzle; and
a controller in communication with the user interface.

19. A method for precleaning a medical instrument via a precleaning process, the method comprising:
providing a point-of-use system including
a housing;
a sanitizing drawer disposed in the housing and configured to accept the medical instrument and to open and close upon activation of a motion sensor disposed on the housing;
a fluid reservoir disposed in the housing and configured to output a cleaning fluid to the sanitizing drawer;
a spray nozzle disposed in the housing and configured to activate automatically after the sanitizing drawer is closed; and
a biohazard container removably disposed in the housing and positioned adjacent the sanitizing drawer, the biohazard container configured to accept the medical instrument from the sanitizing drawer automatically after the medical instrument is precleaned;
activating the motion sensor to open the sanitizing drawer;
placing the medical instrument in the sanitizing drawer;
activating the motion sensor to close the sanitizing drawer;
actuating the spray nozzle to spray the medical instrument with the cleaning fluid; and
transferring the medical instrument from the sanitizing drawer into the biohazard container.

20. The method of claim 19, further including displaying a status of the precleaning process on a user interface.
